(19) United States
(12) Patent Application Publication
(10) Pub. No.: US 2017/0323442 A1
(43) Pub. Date: Nov. 9, 2017

(54) METHOD FOR SUPPORTING A REPORTING PHYSICIAN IN THE EVALUATION OF AN IMAGE DATA SET, IMAGE RECORDING SYSTEM, COMPUTER PROGRAM AND ELECTRONICALLY READABLE DATA CARRIER

(71) Applicant: Siemens Healthcare GmbH, Erlangen (DE)

(72) Inventor: Michael SUEHLING, Erlangen (DE)

(73) Assignee: Siemens Healthcare GmbH, Erlangen (DE)

(21) Appl. No.: 15/654,769

(22) Filed: Jul. 20, 2017

(30) Foreign Application Priority Data
Jul. 22, 2016 (DE) 102016213515.5

(51) Int. Cl. G06T 7/00; A61B 6/00; G06F 19/00; G06N 3/00; G06K 9/62; A61B 6/03

(52) U.S. Cl. CPC G06T 7/0012; G06K 9/6229; A61B 6/566; G06F 19/321; G06F 19/3418; G06N 3/006; G06F 19/3437; A61B 6/032; G06T 2207/10081; G06T 2207/10088; G06T 2207/10132

(57) ABSTRACT

A method for supporting a reporting physician in the evaluation of an image data set of a patient recorded with an image recording system. In an embodiment, the image data set is automatically processed by at least one preprocessing algorithm for display to the reporting physician. In an embodiment, the at least one preprocessing algorithm and/or at least one preprocessing parameter parameterizing the at least one preprocessing algorithm are automatically selected by a selection algorithm of artificial intelligence as a function of at least one item of recording information describing the recording and/or the recording area of the image data set and/or of at least one item of additional information concerning a previous examination of the patient.

### PRIORITY STATEMENT

The present application hereby claims priority under 35 U.S.C. §119 to German patent application number DE 102016213515.5 filed Jul. 22, 2016, the entire contents of which are hereby incorporated herein by reference.

### FIELD

At least one embodiment of the invention generally relates to a method for supporting a reporting physician in the evaluation of an image data set of a patient recorded with an image recording system, wherein the image data set is automatically processed by at least one preprocessing algorithm for display to the reporting physician. In addition, at least one embodiment of the invention generally relates to an image recording system, a computer program and an electronically readable data carrier.

### BACKGROUND

To enable a reporting physician to make an optimum evaluation of image data sets recorded for the examination of a patient, and therefore be able to make reliable diagnoses, preprocessing or pre-evaluation of the image data set is usually provided and useful. To this end, loading the image data set into a clinical application is known today, for example, on a diagnostic workstation computer. The reporting physician can then select dedicated image post-processing algorithms, resulting in significant waiting times, before results are finally available which can be used for further evaluation. This results in a significant loss of performance in the radiology workflow.

In an attempt to solve this problem, designing clinical applications to visualize image data sets has been proposed such that a reporting physician can manually configure rules permitting the selection of special preprocessing algorithms for specific recording information describing the recording and/or the recording area of the image data set, as may be contained, for example, in a DICOM header of the image data set, which are then carried out automatically. Such preprocessing algorithms evaluate the physical and technical conditions which the image data set reproduces in order to improve the image and reproduce information reliably. For example, a preprocessing algorithm can be provided to keep track of vessels in vascular imaging and the like.

### SUMMARY

However, the inventors have discovered that in this approach too, the user must manually identify and specify key phrases in the recording information which trigger special routing and special preprocessing of the image data of the image data set. Such rules are not only extremely cumbersome to configure but also vary between different clinical devices and even users. Indeed, such rules permit recording protocol-specific preprocessing, but not case-specific preprocessing, in other words, preprocessing procedures tailored to an individual patient.

The inventors have discovered that it may often be the case that the same recording protocols and/or recording parameters in general are used, although there is a completely different diagnostic issue. Therefore, even such automation may lead to unsatisfactory results when preparing the evaluation as a result of manually adjusted rules.

At least one embodiment of the invention is therefore to specify improved, completely automated processing of image data sets for diagnosis.

In at least one embodiment, a method enables the at least one preprocessing algorithm and/or at least one preprocessing parameter parameterizing the at least one preprocessing algorithm to be automatically selected by way of a selection algorithm of artificial intelligence as a function of at least one item of recording information describing the recording and/or the recording area of the image data set and/or of at least one item of additional information concerning a previous examination of the patient.

At least one embodiment of the invention therefore proposes a method using artificial intelligence in the form of a selection algorithm to select precisely the preprocessing procedures (“preprocessing”) required wholly without the need for a manual definition of rules and/or any other user intervention, to then also be able to fully automate preprocessing or pre-evaluation, in other words, processing for the reporting physician, and in particular also realize it away from the workstation computer on which the diagnosis takes place.

At least one embodiment of the invention can be realized by a method for supporting a reporting physician in the evaluation of an image data set of a patient recorded with an image recording system, wherein the image data set is automatically processed by at least one preprocessing algorithm for display to the reporting physician. In the method, the at least one preprocessing algorithm and/or at least one preprocessing parameter parameterizing the at least one preprocessing algorithm are automatically selected by a selection algorithm of artificial intelligence as a function of at least one item of recording information describing the recording and/or the recording area of the image data set and/or of at least one item of additional information concerning a previous examination of the patient.

At least one embodiment of the invention can be realized by an image processing system in general which therefore, for example, has a control device which is designed to perform the method according to at least one embodiment of the invention. However, as it is preferable to already perform preprocessing in the image recording system, at least one embodiment of the present invention in particular also relates to an image recording system with a control device which is designed to perform the method according to at least one embodiment of the invention. The control device may have detection units for the recording information which is usually already present on the image recording system, and the additional information, wherein if applicable, corresponding communication devices of the image recording system producing communication connections can be used. In a selection unit, the recording information and the additional information is then analyzed by the selection algorithm in order to deduce corresponding preprocessing steps which are then performed by the preprocessing unit. Thereafter the processed image data set is preferably forwarded to an image archiving system (PACS) which is connected to the image recording system.

At least one embodiment of the invention furthermore relates to a computer program which performs the steps of the method according to at least one embodiment of the invention when it is performed on a computing device, for example, the control device of an image recording system. To this end, the computer program can, for example, be loaded directly into the memory of a control device and has program resources to perform the steps of a method described herein when the program is performed in the control device. The computer program can be stored on an electronically readable data carrier according to at least one embodiment of the invention, which therefore comprises electronically readable control information comprising at least one specified computer program and designed such that it performs a method described herein when the data carrier is used in a control device. The data carrier may be a non-transient data carrier, in particular a CD-ROM.

### BRIEF DESCRIPTION OF THE DRAWINGS

Further advantages and details of the present invention will emerge from the example embodiments described hereinafter and with reference to the diagram. In the figures:

FIG. 1 shows a drawing to explain the method according to an embodiment of the invention,

FIG. 2 shows an illustration of the utilization of the additional information, and

FIG. 3 shows an image processing system in which the method according to an embodiment of the invention can be used.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In at least one embodiment, a method enables the at least one preprocessing algorithm and/or at least one preprocessing parameter parameterizing the at least one preprocessing algorithm to be automatically selected by way of a selection algorithm of artificial intelligence as a function of at least one item of recording information describing the recording and/or the recording area of the image data set and/or of at least one item of additional information concerning a previous examination of the patient.

At least one embodiment of the invention therefore proposes a method using artificial intelligence in the form of a selection algorithm to select precisely the preprocessing procedures (“preprocessing”) required wholly without the need for a manual definition of rules and/or any other user intervention, to then also be able to fully automate preprocessing or pre-evaluation, in other words, processing for the reporting physician, and in particular also realize it away from the workstation computer on which the diagnosis takes place.

Through the additional consideration of additional information available in most cases, which describes the diagnostic issue in more detail in addition to the recording information, automatic, case-specific preprocessing of an image data set is permitted for optimum processing of cases for diagnosis before the image data set is even opened by the reporting physician. Therefore, in addition to semantic recording information, semantic additional information of the patient from previous examinations or procedures is employed to preprocess an image data set optimally. This enables the gap between rudimentary, recording information-specific preprocessing and case-specific preprocessing to be closed such that the quality of the evaluation of the image data set by a reporting physician is improved and it is possible to work more efficiently as it enables a large amount of time to be saved. This is the result of no longer requiring a manual configuration of rules and there also no longer being any waiting time for the subsequent selection of image processing options.

The recording information may, for example, be available stored in a DICOM header of the image data set. The recording information may, for example, involve the specification of a particular recording protocol, it is also conceivable that it explicitly comprises particular recording parameters of the image recording system. It is particularly preferable, however, and this will be discussed in more detail below, if an associated standard is used for the semantic description of the recording information, for example, the so-called RadLex standard, which was established to describe radiology procedures, based on elements which describe an imaging examination, for example, the modality and the body part examined. Standard names and codes for radiology studies are provided for this.

At least one embodiment of the method of the present invention can ultimately be applied to any conceivable medical imaging modality, computer tomography image data sets being discussed more frequently by way of example in the present case. In the case of computer tomography-image recording systems, the recording information may in particular comprise recording protocols/scan protocols, for example, certain organ programs, or the like. Other possible imaging modalities include, for example, magnetic resonance imaging and ultrasound imaging.

An expedient embodiment of the present invention envisages the selection algorithm using a workflow ontology modeling the preprocessing procedure, in which preprocessing information comprising preprocessing algorithms and/or preprocessing parameters and/or from which preprocessing algorithms and/or preprocessing parameters can be derived, is linked to diagnostic information which comprises recording information and/or additional information and/or can be derived from these. Ontology involves a verbally formulated and formally organized representation of a set of notions and the relationships between them in a particular area. Ontologies may also comprise inference and integrity rules, in other words, rules for conclusions and for ensuring their validity, and therefore represent a kind of knowledge representation which can be used particularly advantageously in artificial intelligence.

Therefore, it is also within the scope of the present invention to depict the required preprocessing procedure for the image data set in the form of a workflow ontology, wherein for example, the OWL-S standard can be used. The ontology models preprocessing steps, required input and output information and available preprocessing algorithms and tools such that it contains complete knowledge of the options for preprocessing. Both sequential as well as condition-based workflows may be included in the workflow ontology. The selection algorithm which, for example, can employ semantic reasoning, is then applied to the instance of workflow ontology to derive corresponding preprocessing steps for an available examination.

In particular, provision may be made for the workflow ontology stored on a central computing device, in particular a server, to be accessed by way of a communication connection. In this manner, access to the workflow ontology is enabled for several image processing systems which are designed to perform the method according to at least one embodiment of the invention, wherein furthermore it is possible to constantly expand or update the workflow ontology in a simple manner, as soon as new clinical requirements and/or new options for automated image analysis are known.

The selection algorithm of artificial intelligence can, in general, use statistical information and/or logical dependencies, in particular in the form of inference rules, and/or also be designed as a machine-learning algorithm. The conclusions which the selection algorithm draws can therefore use both statistical information as well as logical dependencies, these being the two main approaches within the scope of artificial intelligence. As algorithms of artificial intelligence, which can also be used within the scope of embodiments of the present invention, have meanwhile become known and described in large numbers in the prior art, this will not be looked at in any detail at this point. Self-learning algorithms which, for example, use training data in which input information is assigned to preprocessing information, are also already known in principle.

After selection of the automatically proposed case-specific preprocessing steps in the form of the preprocessing algorithms and/or preprocessing parameter to be used, the corresponding preprocessing steps are then performed to realize processing as preparation for diagnosis.

In a particularly advantageous embodiment of the present invention, provision is made for the preprocessing algorithms to be performed on a computing device, in particular a control device, of the image-processing device, whereupon the processed image data set is made available to a reporting physician on a workstation computer. It is particularly expedient when the processed image data set is stored in an image archiving system on a dedicated server from where it can be made available to the reporting physician. The option of automatically determining preprocessing steps tailored to the special diagnostic issue within the scope of at least one embodiment of the present invention enables individual use of the image processing capacities of the image recording systems usually extensively available already, to thus also make use of a computing device frequently equipped in this regard already, in particular the control device of the image recording system, for preprocessing and thus relieve other computing devices of an image processing system, in particular the workstation computer provided at the diagnostic workstation, but also the at least one computing device by means of which the image archiving system (PACS) is realized. For it would be extremely complicated and cumbersome to provide special preprocessing routes for different image data sets in one image archiving system before these can finally be stored in processed form in the image archiving system. If preprocessing is performed by the image recording system itself, the data set can be inserted into the image archiving system immediately, already processed for diagnosis, from where it must only be retrieved, processed accordingly, by the reporting physician to undertake diagnosis and evaluation accordingly.

An expedient development of at least one embodiment of the present invention further provides that for recording information and/or additional information at least in part not provided according to a semantic standard especially provided for ontology, the corresponding partial information is converted into the semantic standard by way of semantic analysis, in particular comprising the comparison of textual components. In the workflow ontology, a particular semantic standard is expediently presumed to avoid having to provide different names for each element of the ontology. As, for example, diagnostic reports are frequently drafted in text format, there is not necessarily compliance with such semantic standards. It has been shown, however, that at least with textual components, but in many cases also with others, for example, figurative components, imaging in terms provided by the semantic standard is possible by means of a corresponding semantic analysis. For example, it is feasible to use reference ontologies, wherein for example, freely formulated texts can be searched to be able to find imaging on corresponding semantic concepts. The reference ontologies may correspond to a description of the corresponding semantic standard. Expediently, as already explained, the recording information is at least partially provided in the RadLex standard. This standard was introduced by the Radiological Society of North America (RSNA) as the so-called RadLex Playbook, which is an expansion of RadLex ontology and provides a standardized, comprehensive dictionary of radiology imaging procedures, in particular also semantically defined recording protocols. These semantic recording protocols provide standardized, instantly accessible semantic information by way of an image recording procedure.

It is furthermore preferable when the additional information is at least partially provided in the SNOMED-CT standard and/or in the HL7 standard and/or in the CDA standard and/or as a structured DICOM report. Structured DICOM reports (DICOM SR) frequently include terms from so-called controlled terminologies, for example, SNOMED CT as a semantic standard. Therefore, structured DICOM reports from previous examinations of the patient contain valuable information in a semantically usable format. However, also otherwise, for example, in information systems, reports or diagnostic results are frequently filed in a standardized form, for example, using the HL7-CDA standard, which likewise uses controlled terminologies such as SNOMED CT.

In summary, if both the recording information and the additional information are already available in semantically usable formats, hence using semantic standards, no pre-analysis of this information is necessary, in particular to enable use of the workflow ontology, which is likewise based on these semantic standards.

In an expedient development of at least one embodiment, the additional information can be determined using patient identification information assigned to the image data set and/or contained in the recording information. Frequently, the recording information also already contains patient identification information which alternatively and/or in addition may also be available in the image data set or assigned thereto. This patient identification information enables various sources to be searched for possible existing additional information in order to retrieve this accordingly and to use it for optimum processing of the image data set.

In this context, but also in general, it is expedient when the additional information is at least partially retrieved from an information system, in particular from a hospital information system (HIS) and/or a radiology information system (RIS). If a patient visits the same clinical site several times, for example, a particular hospital and/or a particular radiology practice, the additional information, in other words, reports concerning previous examinations, is usually already assigned to this patient in corresponding information systems, from where it can be retrieved and used. Naturally, it is also conceivable that after the initial registration of a patient at the clinical site in which the image recording system is located, corresponding transfer documents are digitized which are the reason for the examination now undertaken in which the image data set is recorded, such that these may also be present in the information system already.

In principle, it is expedient when a diagnosis which is the reason for the recording of the image data set and/or was made on the basis of a previously, especially at least the immediately previously, recorded image data set is used as additional information. For example, it is frequently provided that structured DICOM reports are filed in an image archiving system together with the corresponding image data set and remain available there. Particularly advantageously, the method according to an embodiment of the invention can be used in all instances in which in any case image data sets are recorded repeatedly in relation to the same treatment/diagnosis, for example, in ontology and/or when planning and/or reviewing interventions, for example, minimally invasive interventions.

Examples of preprocessing algorithms which can be used within the scope of embodiments of the present invention are segmentation algorithms and/or highlighting algorithms and/or measurement algorithms and/or registration algorithms. Naturally, a plurality of further image processing algorithms and their corresponding parameters usable for diagnosis within the scope of the processing of image data sets are also conceivable. Furthermore, within the scope of embodiments of the present invention it may be expedient when useful information, in particular to be displayed with the image data of the image data set and/or referring thereto, is added to the image data set as a function of the additional information by at least one preprocessing algorithm. For example, this may comprise scales, bases for evaluation and the like.

If in an example a computer tomography image data set of the abdomen of a patient is to be preprocessed, it may possibly be concluded from the additional information that the patient is suffering from colon cancer which has already been diagnosed. It is now known, for example, on the basis of corresponding relationships in the workflow ontology, that such colon cancer frequently spreads to the liver, from which it can in turn be concluded that the liver is a relevant object of examination with regard to metastases. Corresponding preprocessing steps can be taken, for example, corresponding segmentation procedures, highlighting procedures, measurements, the addition of useful information such as size charts and the like, etc. All this takes place completely automatically and expediently before the transmission of the image data set to the image archiving system, such that it is available there already completely processed and ready for diagnosis.

The method according to at least one embodiment of the invention can be realized by an image processing system in general which therefore, for example, has a control device which is designed to perform the method according to at least one embodiment of the invention. However, as it is preferable to already perform preprocessing in the image recording system, at least one embodiment of the present invention in particular also relates to an image recording system with a control device which is designed to perform the method according to at least one embodiment of the invention. The control device may have detection units for the recording information which is usually already present on the image recording system, and the additional information, wherein if applicable, corresponding communication devices of the image recording system producing communication connections can be used. In a selection unit, the recording information and the additional information is then analyzed by the selection algorithm in order to deduce corresponding preprocessing steps which are then performed by the preprocessing unit. Thereafter the processed image data set is preferably forwarded to an image archiving system (PACS) which is connected to the image recording system.

At least one embodiment of the invention furthermore relates to a computer program which performs the steps of the method according to at least one embodiment of the invention when it is performed on a computing device, for example, the control device of an image recording system. To this end, the computer program can, for example, be loaded directly into the memory of a control device and has program resources to perform the steps of a method described herein when the program is performed in the control device. The computer program can be stored on an electronically readable data carrier according to at least one embodiment of the invention, which therefore comprises electronically readable control information comprising at least one specified computer program and designed such that it performs a method described herein when the data carrier is used in a control device. The data carrier may be a non-transient data carrier, in particular a CD-ROM.

Example embodiments of the method according to the invention permit appropriate preprocessing steps of a preprocessing procedure (preprocessing) to be selected and performed completely automatically for an image data set, which prepare this especially for the desired diagnostic issue. The example embodiment of the method according to the invention described hereinafter takes place in the control device of the image recording system itself, such that the image data set already processed can be forwarded to the image archiving system (PACS).

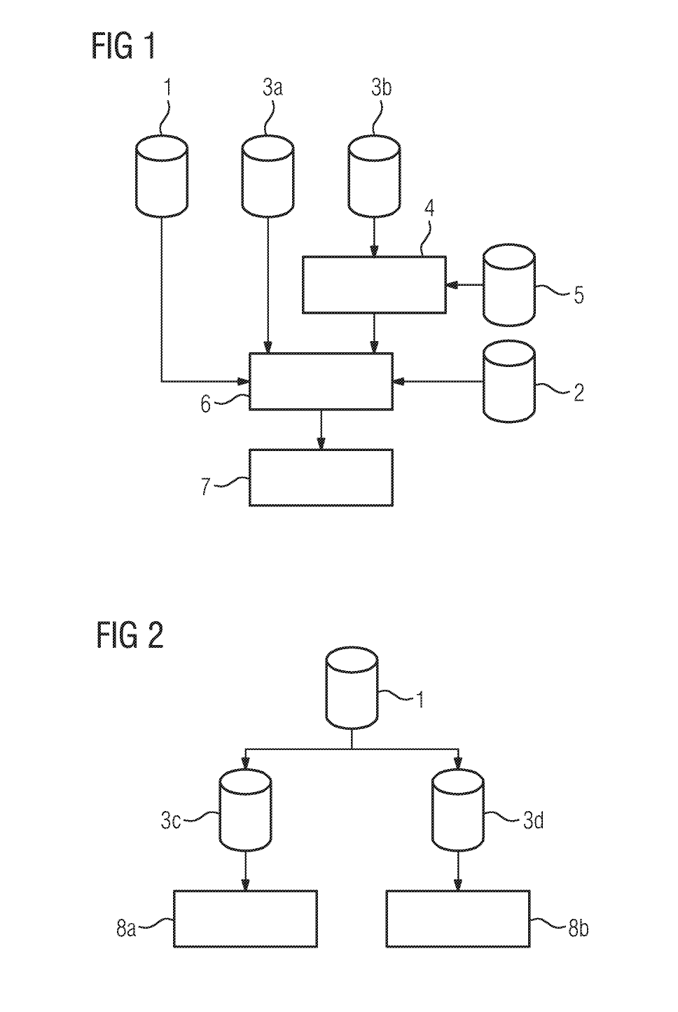

As input data, the method first uses, cf. FIG. 1, recording information 1 which is already available on the part of the image recording system. The recording information 1 is available according to the RadLex Playbook of the Radiological Society of North America (RSNA), therefore in a semantic standard which complies with that in a workflow ontology 2 used by artificial intelligence, which will be described in more detail hereinafter.

The recording information 1 in this case also comprises patient identification information which can be used to retrieve additional information 3a, 3b from various other sources accessible by way of communication connections. The additional information relates to previous examinations, in particular their diagnostic results, of the same patient. The additional information 3a comprises additional information assigned to structured DICOM reports in an image archiving system (PACS) in the form of assigned previous image data sets, wherein semantic standards of the workflow ontology 2 are observed such that if necessary after an extraction of the relevant parts, the additional information 3a is likewise immediately usable.

The situation is different with the additional information 3b, which in the present case is retrieved from an information system, for example, a hospital information system (HIS) or a radiology information system (RIS). This involves previous diagnostic reports in text format which do not necessarily meet the semantic standards on which the workflow ontology 2 is based. Therefore, a semantic analysis takes place in a step 4 for the corresponding additional information 3b, wherein in particular textual components are compared with those in a reference ontology 5 which ultimately complies with the semantic standard which is used in the workflow ontology 2. As semantic standards for the additional information, in addition to the aforementioned RadLex standard, SNOMED CT, HL7 and CDA can be used.

In a step 6, the recording information 1, the additional information 3a and the additional information 3b described in corresponding semantic standards are added to a selection algorithm of the artificial intelligence which evaluates it using the workflow ontology 2 which establishes links to preprocessing information. Semantic reasoning is preferably used in the selection algorithm, wherein statistical information can be used in the same way as logical dependencies to ultimately deduce specific preprocessing steps of a processing procedure which use certain preprocessing algorithms and/or preprocessing parameters. The selection algorithm can be a learning algorithm.

After the additional information 3a, 3b is likewise taken into consideration, case-specific preprocessing occurs and thus processing of the image data set for the following diagnosis as the concrete diagnostic issue can be deduced. The determined preprocessing steps are also performed accordingly by the control device of the image recording system in a step 7, whereupon the image data set preprocessed in this way is forwarded to the image archiving system, where it is available for diagnosis.

FIG. 2 depicts a significant advantage of the method according to an embodiment of the invention in the form of a schematic drawing. It essentially involves two different patients with two different diagnostic issues to respond to which, however, the same computer tomography recording protocol, described by the same recording information 1, is used, in this specific example a two-phase computer tomography scan of the abdomen using contrast agent. However, as additional information is also used, here for the first patient the additional information 3c and for the second patient the additional information 3d, it is semantically clear from these that the first patient is suffering from colon cancer and that the image data set is a follow-up examination during chemotherapy. However, the second patient who, as emerges semantically from the additional information 3d, is suffering from an aneurysm of the aorta (AAA), is examined after an Endovascular Aortic Aneurysm Repair (EVAR) has been performed.

This now results in completely different preprocessing steps 8a and 8b for the two patients, when the selection algorithm of artificial intelligence in step 6 is used. Thus, the preprocessing steps 8a for the first patient may comprise:
Advance data sets recorded previously are retrieved from the image archiving system and the items of image data are registered with each other to be able to see them side by side during diagnosis,
A lesion CAD algorithm for detecting metastases in primary scattering centers is performed as a preprocessing algorithm, wherein the typical scatter areas comprise the liver, the lungs and the peritoneum,
Detected lesions in previously recorded advance data sets and the current image data set are segmented and changes in the size of the lesions are precalculated,
Bone-organ-development algorithms are performed to support the reporting physician in detecting metastases,
TNM classification guidelines for colon cancer are added to the image data set as useful information to be displayed for diagnosis when the image data set is retrieved, and
Colon cancer therapy guidelines are likewise added as useful information for display during diagnosis.

In contrast, the preprocessing steps 8b for the second patient or their image data set comprise:
The aorta is automatically tracked and visualized,
Aneurysms in the aorta are automatically detected and segmented,
An already inserted stent is appropriately visualized,
Typical complications after the intervention, for example, plaque embolism, endoleaks and the like, are automatically detected, and
Guidelines for classification of endoleaks are added as useful information in the image data set and displayed on retrieval for diagnosis.

In spite of the use of the same imaging technology and in particular also the same image acquisition parameters, evidently the additional information also permits the differentiation of completely different diagnostic issues and the automatic realization of corresponding preprocessing.

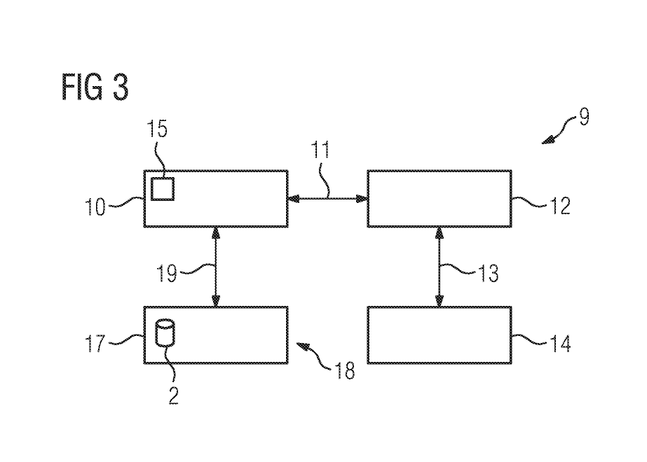

FIG. 3 shows a schematic diagram of an image processing system 9 in which the method according to an embodiment of the invention can be performed. The image processing system 9 comprises at least one image recording system 10, for example, a computer tomography image recording system. This has a communication connection 11 with an image archiving system 12 (PACS) in which image data sets can be filed. By way of a further communication connection 13 which can be realized by way of the same network as the communication connection 11, workstation computers 14 can access diagnostic workstations on the image archiving system 12.

Preferably the method according to an embodiment of the invention is performed by a control device 15 of the image recording system 10 such that the preprocessed image data set already processed can be inserted into the image archiving system 12. In this way, the calculation requirements and waiting times are significantly reduced for the workstation computer 14.

As part of the image processing system 9, a central computing device 17 can also be provided, which can implement an information system 18, for example, an HIS or an RIS, and/or can be filed on the workflow ontology 12 for retrieval and/or access by several image recording systems, cf. arrow 19. The central storage of the workflow ontology 2 enables simple updating and/or expansion. It is pointed out that preprocessing algorithms newly added to the workflow ontology 2 can also be kept on the central computing device 17, which is here designed as a server, for retrieval by the control device 15 such that corresponding preprocessing steps can also be effectively performed when these have been determined by the selection algorithm of artificial intelligence in step 6.

Although the invention was illustrated and described in more detail by the preferred example embodiment, the invention is not restricted by the disclosed examples and other variations can be deduced by a person skilled in the art without departing from the scope of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for supporting a reporting physician in evaluation of an image data set of a patient recorded with an image recording system, the method comprising:
automatically selecting at least one of at least one preprocessing algorithm for display to the reporting physician and at least one preprocessing parameter parameterizing the at least one preprocessing algorithm, via a selection algorithm of artificial intelligence, as a function of at least one item of recording information describing at least one of recording and a recording area of at least one of the image data set and at least one item of additional information concerning a previous examination of the patient.

2. The method of claim 1, wherein the selection algorithm uses a workflow ontology modeling a preprocessing procedure, in which preprocessing information comprising at least one of the at least one preprocessing algorithm and the at least one preprocessing parameters and/or from which the at least one preprocessing algorithm and the at least one preprocessing parameter is derivable, and linked to diagnostic information comprising at least one of recording information, additional information and information derivable from at least one of the recording information and the additional information.

3. The method of claim 1, wherein the at least one preprocessing algorithm is performed on a control device of the image recording system, and wherein thereafter, the image data set is made available to a reporting physician on a workstation computer.

4. The method of claim 3, wherein the image data set, after processing, is stored in an image archiving system on an assigned server and thereafter made available to the reporting physician.

5. The method of claim 1, further comprising:
converting, for at least one of available recording information and additional information and at least partially not in accordance with a semantic standard provided for workflow ontology, corresponding partial information via semantic analysis, into the semantic standard.

6. The method of claim 1, wherein at least one of
the recording information is provided at least partially in the RadLex standard and
the additional information is provided at least partially at least one of in the SNOMED CT standard, in the HL7 standard, in the CDA standard and as a structured DICOM report.

7. The method of claim 1, wherein at least one of
the additional information is determined using patient identification information at least one of assigned to the image data set and contained in the recording information,
the additional information is at least partially retrieved from at least one of an information system and a diagnosis giving rise to the recording of the image data set, and
a previously recorded image data set is used as the additional information.

8. The method of claim 1, wherein at least one of
the selection algorithm of artificial intelligence uses at least one of statistical information and logical dependencies, in particular in the form of inference rules, and
the selection algorithm is a machine-learning algorithm.

9. The method of claim 1, wherein the at least one preprocessing algorithm comprises at least one of segmentation algorithms, highlighting algorithms, measurement algorithms and registration algorithms.

10. An image recording system comprising:
a control device, configured to:
automatically select at least one of at least one preprocessing algorithm for display to a reporting physician and at least one preprocessing parameter parameterizing the at least one preprocessing algorithm, via a selection algorithm of artificial intelligence, as a function of at least one item of recording information describing at least one of recording and a recording area of at least one of the image data set and at least one item of additional information concerning a previous examination of the patient.

11. A non-transitory computer program including program code for carrying out the method of claim 1 when the program code is run on a computing device.

12. A non-transitory electronically readable data carrier including program code for carrying out the method of claim 1 when the program code is run in a computer.

13. The method of claim 2, wherein the at least one preprocessing algorithm is performed on a control device of the image recording system, and wherein thereafter, the image data set is made available to a reporting physician on a workstation computer.

14. The method of claim 13, wherein the image data set, after processing, is stored in an image archiving system on an assigned server and thereafter made available to the reporting physician.

15. The method of claim 5, wherein the converting of corresponding partial information via semantic analysis includes comparing of textual components.

16. The method of claim 7, wherein the additional information being at least partially retrieved from an information system includes at least partially retrieving the additional information from a hospital information system and wherein the previously recorded image data set is at least the image data set recorded immediately before.

17. The method of claim 8, wherein the at least one of statistical information and logical dependencies are in the form of inference rules.

18. The method of claim 9, wherein useful information is added to the image data set by the at least one preprocessing algorithm.

19. The method of claim 18, wherein useful information, to be displayed with the image data of the image data set as a function of the additional information, is added to the image data set by the at least one preprocessing algorithm.

20. The image recording system of claim 10, wherein the selection algorithm uses a workflow ontology modeling a preprocessing procedure, in which preprocessing information comprising at least one of the at least one preprocessing algorithm and the at least one preprocessing parameters and/or from which the at least one preprocessing algorithm and the at least one preprocessing parameter is derivable, and linked to diagnostic information comprising at least one of recording information, additional information and information derivable from at least one of the recording information and the additional information.

21. The image recording system of claim 10, wherein the at least one preprocessing algorithm is performed on the control device of the image recording system, and wherein thereafter, the image data set is made available to a reporting physician on a workstation computer.

22. The image recording system of claim 10, wherein the image data set, after processing, is stored in an image archiving system on an assigned server and thereafter made available to the reporting physician.
